SEQUENCE LISTING (51 sequences)

SEQ ID NO: 1 - DNA, 47 bp, Artificial Sequence
  Description of Artificial Sequence Synthetic oligonucleotide
cggatagtgttctttcgctagaccatgtgacgcatggtgagatgctt

SEQ ID NO: 2 - DNA, 55 bp, Artificial Sequence
  Description of Artificial Sequence Synthetic oligonucleotide
cggatagtgttctttcgctagactgggggagtattgcggaggaagtgagatgctt

SEQ ID NO: 3 - DNA, 13 bp, Artificial Sequence
  Description of Artificial Sequence Synthetic oligonucleotide
aagcatctcaagc

SEQ ID NO: 4 - DNA, 13 bp, Artificial Sequence
  Description of Artificial Sequence Synthetic oligonucleotide
ggaacactatccg

SEQ ID NO: 5 - DNA, 13 bp, Artificial Sequence
  Description of Artificial Sequence Synthetic oligonucleotide
cggatagtgttcc

SEQ ID NO: 6 - DNA, 13 bp, Artificial Sequence
  Description of Artificial Sequence Synthetic oligonucleotide
gcttgagatgctt

SEQ ID NO: 7 - DNA, 21 bp, Artificial Sequence
  Description of Artificial Sequence Synthetic aptamer motif sequence
gggttagggttagggttaggg

SEQ ID NO: 8 - RNA, 29 nt, Artificial Sequence
  Description of Artificial Sequence Synthetic aptamer motif sequence
aggcgaggugaaaugagcgguaauagccu

SEQ ID NO: 9 - RNA, 95 nt, Artificial Sequence
  Description of Artificial Sequence Synthetic aptamer motif sequence
gggagaggauacuacacgugauagucagggaacaugacaaacacagggacuugcgaaaaucaguguuuugccauugcauguagcagaagcuuccg

SEQ ID NO: 10 - DNA, 132 bp, Artificial Sequence
  Description of Artificial Sequence Synthetic aptamer motif sequence
gggagaattcccgcggcagaagcccacctggctttgaactctatgttattgggtgggggaaacttaagaaaactaccacccttcaacattaccgcccttcagcctgccagcgccctgcagcccgggaagctt

SEQ ID NO: 11 - RNA, 38 nt, Artificial Sequence
  Description of Artificial Sequence Synthetic aptamer motif sequence
ggaucccgacuggcgagagccagguaacgaauggaucc

SEQ ID NO: 12 - DNA, 29 bp, Artificial Sequence
  Description of Artificial Sequence Synthetic aptamer motif sequence
ccggccaagggtgggagggagggggccgg

SEQ ID NO: 13 - RNA, 61 nt, Artificial Sequence
  Description of Artificial Sequence Synthetic aptamer motif sequence
auggcaccgaccauaggcucggguugccagagguuccacacuuucaucgaaaagccuaugc

SEQ ID NO: 14 - RNA, 33 nt, Artificial Sequence
  Description of Artificial Sequence Synthetic aptamer motif sequence
ggcgauaccagccgaaaggcccuuggcagcguc

SEQ ID NO: 15 - RNA, 52 nt, Artificial Sequence
  Description of Artificial Sequence Synthetic aptamer motif sequence
gauaggacgauuaucgaaaaucaccagauuggacccugguuaacgauccauu

SEQ ID NO: 16 - DNA, 30 bp, Artificial Sequence
  Description of Artificial Sequence Synthetic aptamer motif sequence
gacaaggaaaatccttcaatgaagtgggtc

SEQ ID NO: 17 - RNA, 67 nt, Artificial Sequence
  Description of Artificial Sequence Synthetic aptamer motif sequence
gggaauuccgcgugugcgccgcggaagagggaauauagaggccagcacauagugaggcccuccuccc

SEQ ID NO: 18 - RNA, 72 nt, Artificial Sequence
  Description of Artificial Sequence Synthetic aptamer motif sequence
gggagcucagaauaaacgcucaaggaggaccgugcacuccucgaacauuucgagaugagacacggauccugc

SEQ ID NO: 19 - RNA, 44 nt, Artificial Sequence
  Description of Artificial Sequence Synthetic aptamer motif sequence
gacgagaaggagugcugguuauacuagcgguuaggucacucguc

SEQ ID NO: 20 - DNA, 27 bp, Artificial Sequence
  Description of Artificial Sequence Synthetic aptamer motif sequence
acctgggggagtattgcggaggaaggt

SEQ ID NO: 21 - RNA, 31 nt, Artificial Sequence
  Description of Artificial Sequence Synthetic aptamer motif sequence
ggaagagauggcgacuaaaacgacuugucgc

SEQ ID NO: 22 - RNA, 39 nt, Artificial Sequence
  Description of Artificial Sequence Synthetic aptamer motif sequence
ucuagcaguucagguaaccacguaagauacgggucuaga

SEQ ID NO: 23 - RNA, 84 nt, Artificial Sequence
  Description of Artificial Sequence Synthetic aptamer motif sequence
gggagcucagaauaaacgcucaacccgacagaucggcaacgccnuguuuucgacangagacaccgauccugcaccaaagcuucc

SEQ ID NO: 24 - DNA, 65 bp, Artificial Sequence
  Description of Artificial Sequence Synthetic aptamer motif sequence
gcagtctcgtcgacacccagcagcgcatgtaactcccatacatgtgtgtgctggatccgacgcag

SEQ ID NO: 25 - RNA, 52 nt, Artificial Sequence
  Description of Artificial Sequence Synthetic aptamer motif sequence
gggcacgagcgaagggcauaagcugacgaaagucagacaagacauggugccc

SEQ ID NO: 26 - RNA, 49 nt, Artificial Sequence
  Description of Artificial Sequence Synthetic aptamer motif sequence
ggaacccaacuaggcguuugaggggauucggccacgguaacaaccccuc

SEQ ID NO: 27 - RNA, 45 nt, Artificial Sequence
  Description of Artificial Sequence Synthetic aptamer motif sequence
gggcauaagguauuuaauuccauacaaguuuacaagaaagaugca

SEQ ID NO: 28 - DNA, 37 bp, Artificial Sequence
  Description of Artificial Sequence Synthetic aptamer motif sequence
taaactaaatgtggagggtgggacgggaagaagttta

SEQ ID NO: 29 - RNA, 31 nt, Artificial Sequence
  Description of Artificial Sequence Synthetic aptamer motif sequence
ccggugcgcauaaccaccucagugcgagcaa

SEQ ID NO: 30 - RNA, 108 nt, Artificial Sequence
  Description of Artificial Sequence Synthetic aptamer motif sequence
gggagaauuccgaccagaagcuuugguugucuuguacguucacuguuacgauuguguuagguuuaacuacacuuugcaaucgcauaugugcgucuacauggauccuca

SEQ ID NO: 31 - DNA, 41 bp, Artificial Sequence
  Description of Artificial Sequence Synthetic aptamer motif sequence
gcggggttgggcgggtgggttcgctgggcagggggcgagtg

SEQ ID NO: 32 - RNA, 40 nt, Artificial Sequence
  Description of Artificial Sequence Synthetic aptamer motif sequence
uacagaauggguugguaggcauaccuaaucgagaaugaua

SEQ ID NO: 33 - RNA, 113 nt, Artificial Sequence
  Description of Artificial Sequence Synthetic aptamer motif sequence
ggagcucagccuucacugcaaugggccgcuagguugaugugcagugaagucagcugaggcccagggcugaaaggaucgcccuccucgacucguggcaccacggucggauccac

SEQ ID NO: 34 - RNA, 45 nt, Artificial Sequence
  Description of Artificial Sequence Synthetic aptamer motif sequence
ggaucgcauuuggacuucugcccagggggcaccacggucggaucc

SEQ ID NO: 35 - RNA, 60 nt, Artificial Sequence
  Description of Artificial Sequence Synthetic aptamer motif sequence
ggccuaaaacauaccagauuucgaucuggagaggugaagaauucgaccaccuaggccggu

SEQ ID NO: 36 - DNA, 46 bp, Artificial Sequence
  Description of Artificial Sequence Synthetic aptamer motif sequence
acgtgaatgatagacgtatgtcgagttgctgtgtgcggatgaacgt

SEQ ID NO: 37 - RNA, 107 nt, Artificial Sequence
  Description of Artificial Sequence Synthetic aptamer motif sequence
gggagcugagaauaaacgcucaagggcaacgcgggcaccccgacaggugcaaaaacgcaccgacgcccggccgaagaaggggauucgacaugaggcccggauccggc

SEQ ID NO: 38 - RNA, 24 nt, Artificial Sequence
  Description of Artificial Sequence Synthetic aptamer motif sequence
uccguuuucagucgggaaaaacug

SEQ ID NO: 39 - DNA, 15 bp, Artificial Sequence
  Description of Artificial Sequence Synthetic aptamer motif sequence
ggttggtgtggttgg

SEQ ID NO: 40 - RNA, 23 nt, Artificial Sequence
  Description of Artificial Sequence Synthetic aptamer motif sequence
gcgguaggaagaauuggaagcgc

SEQ ID NO: 41 - RNA, 98 nt, Artificial Sequence
  Description of Artificial Sequence Synthetic aptamer motif sequence
gggauauccucgagacauaagaaacaagauagauccugaaacuguuuuaagguuggccgaucuucugcucgagaaugcaugaagcguuccauauuuuu

SEQ ID NO: 42 - DNA, 37 bp, Artificial Sequence
  Description of Artificial Sequence Synthetic aptamer motif sequence
ggggcacgtttatccgtccctcctagtggcgtgcccc

SEQ ID NO: 43 - RNA, 44 nt, Artificial Sequence
  Description of Artificial Sequence Synthetic aptamer motif sequence
ggggcuauugugacucagcgguucgaccccgcuuagcuccacca

SEQ ID NO: 44 - RNA, 39 nt, Artificial Sequence
  Description of Artificial Sequence Synthetic aptamer motif sequence
ugacguccuuagaauugcgcauuccucacacaggaucuu

SEQ ID NO: 45 - DNA, 96 bp, Artificial Sequence
  Description of Artificial Sequence Synthetic aptamer motif sequence
ataccagcttattcaattaggcggtgcattgtggttggtagtatacatgaggtttggttgagactagtcgcaagatatagatagtaagtgcaatct

SEQ ID NO: 46 - RNA, 28 nt, Artificial Sequence
  Description of Artificial Sequence Synthetic aptamer motif sequence
aggacccucgagggagguugcgcagggu

SEQ ID NO: 47 - DNA, 26 bp, Artificial Sequence
  Description of Artificial Sequence Synthetic oligonucleotide
aagcatctcaagcggaacactatccg

SEQ ID NO: 48 - DNA, 47 bp, Artificial Sequence; identical to SEQ ID NO: 1
  Description of Artificial Sequence Synthetic oligonucleotide

SEQ ID NO: 49 - DNA, 26 bp, Artificial Sequence
  Description of Artificial Sequence Synthetic oligonucleotide
aagcatctcaagcggaacactatccg

SEQ ID NO: 50 - DNA, 55 bp, Artificial Sequence; identical to SEQ ID NO: 2
  Description of Artificial Sequence Synthetic oligonucleotide

SEQ ID NO: 51 - DNA, 29 bp, Artificial Sequence
  Description of Artificial Sequence Synthetic oligonucleotide
accatgcgtcacatggtctagcgaaagaa

(19) United States
(12) Patent Application Publication
(10) Pub. No.: US 2006/0166222 A1
(43) Pub. Date: Jul. 27, 2006

(54) Nucleic acid enzyme ligation sensor

(76) Inventors: Yi Lu, Champaign, IL (US); Juewen Liu, Urban, IL (US)

Correspondence Address: EVAN LAW GROUP LLC, 566 WEST ADAMS, SUITE 350, CHICAGO, IL, 60661, US

(21) Appl. No.: 11/041,003

(22) Filed: Jan. 21, 2005

(51) Int. Cl. C12Q 1/68; C12P 19/34; C12M 1/34

(52) U.S. Cl. 435/6; 435/91.2; 435/287.2

(57) ABSTRACT

The present invention provides a calorimetric light-up sensor for determining the presence and optionally the concentration of an analyte in a sample. Methods of utilizing the sensor and kits that include the sensor also are provided. The sensor utilizes nucleic acid enzymes that ligate substrate fragments to form aggregates from the resulting substrate and oligonucleotide functionalized particles. The nucleic acid enzymes useful in the ligation may include DNA and RNA based enzymes and enzymes modified with aptamer components to form aptazymes.

### FEDERALLY SPONSORED RESEARCH OR DEVELOPMENT

This subject matter of this application may have been funded in part under the following research grants and contracts: DOE Grant No. DEFG02-01-ER63179, NSF CTS-0120978, NSF DMR-0117792, and HUD ILLTS0097-03. The U.S. Government may have rights in this invention.

### BACKGROUND

The ability to determine the presence of an analyte in a sample is of significant benefit. For example, many metals and metal ions, such as lead, mercury, cadmium, chromium, and arsenic, pose significant health risks when present in drinking water supplies. To prevent the contamination of drinking and other water supplies, it is common to test industrial waste-streams before their release to the water treatment plant. For example, waste-streams from metal plating operations routinely contain undesirable or biologically harmful concentrations of copper and zinc. Biological fluids, such as blood and those originating from body tissues, also may be tested for a variety of analytes to determine if the body has been exposed to harmful agents or if a disease state exists. For example, recently there has been the need to detect trace amounts of anthrax and other biologically harmful agents in a variety of samples.

Colorimetric sensor systems are commonly used for the detection of metals and ions in soil, water, waste-streams, biological samples, body fluids, and the like. In relation to instrument based methods of analysis, such as atomic absorption spectroscopy, colorimetric methods tend to be rapid and require little in the way of equipment or user sophistication. For example, calorimetric sensors are available to aquarists that turn darker shades of pink when added to aqueous samples containing increasing concentrations of the nitrate (NO₃⁻) ion. In this manner, colorimetric sensors show that the analyte of interest, such as nitrate, is present in the sample and also may provide an indicator of the amount of analyte in the sample through the specific hue of color generated.

While colorimetric sensor systems are extremely useful, they only exist for a limited set of analytes, often cannot detect very small or trace amounts of the analyte, and depending on the nature of the sample, can generate unacceptable levels of false positive or negative results. False positives occur when the calorimetric reagents produce the color associated with the presence of an analyte when the analyte is not present, while false negatives occur when the analyte of interest is present in the sample, but the expected color is not produced. False positives are often the result of constituents in the sample that the calorimetric test cannot distinguish from the analyte of interest. Related to false positives are background levels of color change not associated with the analyte of interest. These background color changes reduce the sensitivity of the sensor system to the analyte. Thus, for the sensor to detect the analyte, enough of the analyte must be present in the sample to distinguish the color change response to the analyte from that associated with the background.

As can be seen from the above description, there is an ongoing need for colorimetric tests that can identify trace amounts of a broader scope of analytes. Furthermore, colorimetric tests having a lower incidence of false positives and increased sensitivity also would provide significant benefit.

### SUMMARY

In one aspect of the invention, a sensor system is disclosed that includes a ligase, a plurality of substrate fragments, and first particles. The substrate fragments may include first polynucleotides and the first particles may include second polynucleotides that are coupled to the first particles. The first polynucleotides may be at least partially complementary to the second polynucleotides. At least two of the substrate fragments may undergo ligation in the presence of the analyte. The sensor system also may include second particles that include third polynucleotides that are at least partially complementary to the fourth polynucleotides.

In another aspect of the invention, a method of detecting an analyte is disclosed that includes combining a sample and a plurality of substrate fragments with first particles to detect a color change responsive to the analyte. The plurality of substrate fragments includes first polynucleotides that are at least partially complementary to the second polynucleotides and the first particles include second polynucleotides coupled to the first particles. At least two of the substrate fragments undergo ligation in the presence of the analyte.

In another aspect of the invention, a kit for detecting an analyte is disclosed that includes a first container containing a system for forming aggregates. The system includes a plurality of substrate fragments where at least two of the fragments of the plurality undergo ligation in the presence of the analyte. Also included in the system are first particles

In order to provide a clear and consistent understanding of the specification and claims, the following definitions are provided.

The term “sample” or “test sample” is defined as a composition that will be subjected to analysis that is suspected of containing the analyte of interest. Typically, a sample for analysis is in a liquid form, and preferably the sample is an aqueous mixture. A sample may be from any source, such as an industrial sample from a waste-stream or a biological sample, such as blood, urine, or saliva. A sample may be a derivative of an industrial or biological sample, such as an extract, a dilution, a filtrate, or a reconstituted precipitate.

The term “analyte” is defined as one or more substance potentially present in the sample. The analysis process determines the presence, quantity, or concentration of the analyte present in the sample.

The term “calorimetric” is defined as an analysis process where the reagent or reagents constituting the sensor system produce a color change in the presence or absence of an analyte.

The term “sensitivity” refers to the lower concentration limit at which a sensor system can detect an analyte. Thus, the more sensitive a sensor system is to an analyte, the better the system is at detecting lower concentrations of the analyte.

The term “selectivity” refers to the ability of the sensor system to detect the desired analyte in the presence of other species.

The term “hybridization” refers to the ability of a first polynucleotide to form at least one hydrogen bond with at least one second nucleotide under low stringency conditions.

The term “ligase” refers to a moiety capable of joining two or more substrate fragments. For example, ligases may include nucleic acid enzymes, protein enzymes, small molecule mimics of ligase enzymes, and the like.

### BRIEF DESCRIPTION OF THE DRAWINGS

The invention can be better understood with reference to the following drawings and description. The components in the figures are not necessarily to scale and are not intended to accurately represent molecules or their interactions, emphasis instead being placed upon illustrating the principles of the invention.

FIG. 1 represents a calorimetric analytic method of determining the presence and optionally the concentration of an analyte in a sample.

FIG. 2A represents a DNAzyme that depends on Zn(II) and/or Cu(II) as a co-factor to display catalytic activity.

FIG. 2B represents a DNA aptazyme that was formed from a DNAzyme through replacement of the hairpin structure by an aptamer motif that selectively binds adenosine.

FIG. 2C is a graph depicting ligation as a function of time in the presence of the aptamer target for the DNA aptazyme depicted in FIG. 2B.

FIG. 3A represents the ligation of substrate fragments to form a ligation product by a DNAzyme.

FIG. 3B represents the ligation of substrate fragments to form a ligation product by a DNA aptazyme.

FIG. 3C represents the formation of a ligation product by a DNAzyme and an aggregate in the presence of Zn(II) and Cu(II) analytes.

FIG. 4A is a graph showing that the fraction of substrate fragments ligated increased with time in the presence of 1 mM of Zn(II).

FIG. 4B shows the time-dependent change of ligated and unligated substrate fragments in the presence of 0.1 mM of Cu(II) and 1 mM of Zn(II).

FIG. 5A is a graph showing the extinction peaks for oligonucleotide functionalized gold nanoparticles in the presence (dashed line) or absence (solid line) of a ligation product.

FIG. 5B is a graph showing the extinction coefficients at 260 nm of the aggregated nanoparticles as temperature is increased.

FIG. 6 is a graph depicting the change in extinction ratios for oligonucleotide functionalized gold nanoparticles for various concentrations of Cu(II) and Zn(II).

FIG. 7A is a graph depicting the extinction ratios for Cu(II), Zn(II), Mn(II), Co(II), Ni(II), Ca(II), Pb(II), and Cd(II) at the 0.01, 0.1, and 1 mM solution concentrations.

FIG. 7B depicts the extinction ratios for a sample containing a mixture of metal ions.

### DETAILED DESCRIPTION

In related applications, such as U.S. Ser. No. 10/144,679, filed May 10, 2002, entitled “Simple catalytic DNA biosensors for ions based on color changes,” colorimetric sensors were disclosed that utilized a Nucleic Acid Enzyme (NAE) to cleave a substrate, thus providing for the disaggregation of an aggregate. In these prior sensor systems, a sample was added to a DNAzyme/Substrate/particle aggregate, where the substrate was cleaved if the sample included the analyte. The resultant disaggregation brought about a color change that signified the presence of the analyte.

False positives are possible for this and other cleavage-based sensors due to cleavage occurring without the presence of the analyte. Such undesired cleavage without the analyte may be referred to as background cleavage and may reduce the sensitivity of the sensor by providing color change that is not responsive to the concentration of the analyte.

The present invention makes use of the discovery that by replacing the NAE capable of cleaving a substrate with a ligase capable of joining two substrate fragments (ligation) the disadvantage of undesired background cleavage may be reduced. In this manner, the sensitivity of the present sensor to the one or more analytes may be improved through a reduction in the background color change. Thus, a calorimetric sensor is provided that undergoes the desired color change in response to an analyte at room temperature with reduced background ligation.

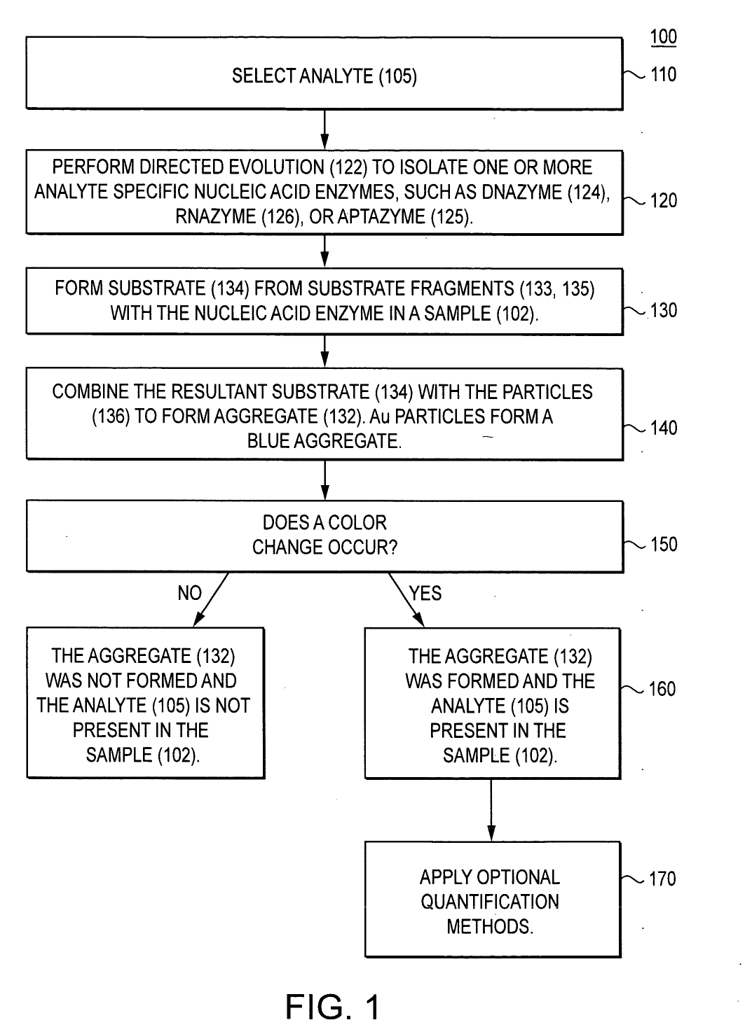

FIG. 1 represents a colorimetric analytic method 100 of determining the presence and optionally the concentration of an analyte 105 in a sample 102 (not shown). In 110, the analyte 105 for which the method 100 will determine the presence/concentration of is selected.

In one aspect, the analyte 105 may be any ion that can serve as a co-factor for a ligation reaction, as discussed further below. Preferable monovalent metal ions having a ⁺1 formal oxidation state (I) include Li(I), TI(I), and Ag(I). Preferable divalent metal ions having a ⁺2 formal oxidation state (II) include Mg(II), Ca(II), Mn(II), Co(II), Ni(II), Zn(II), Cd(II), Cu(II), Pb(II), Hg(II), Pt(II), Ra(II), Sr(II), Ni(II), and Ba(II). Preferable trivalent and higher metal ions having ⁺3 (III), ⁺4 (IV), ⁺5 (V), or ⁺6 (VI) formal oxidation states include Co(III), Cr(IIIl), Ce(IV), As(V), U(VI), Cr(VI), and lanthanide ions. More preferred analyte ions include Zn(II), Cu(II), Ag(I), Pb(II), Hg(II), U(VI), and Cr(VI) due to the desire to eliminate these ions from water supplies. At present, especially preferred analyte ion are Zn(II) and Cu(II).

The analyte 105 also may be in the form of metal ions or non-metal ions and other molecules that bind with a specific aptamer motif, such as K(I), Zn(II), Ni(II), organic dyes, biotin, theophylline, adenine, dopamine, amino acids, nucleosides/nucleotides, RNA, biological co-factors, amino-glycosides, oligosaccharides, polysaccharides, peptides, enzymes, growth factors, transcription factors, antibodies, gene regulatory factors, cell adhesion molecules, cells, viral components, bacterial components, NH₄⁺, spermine, spermidine, adenosine, HIV, HIV proteins, HIV-derived molecules, anthrax, anthrax-derived molecules, small pox, small pox-derived molecules, nitrogen fertilizers, pesticides, dioxins, phenols, 2,4-dichlorophenoxyacetic acid, nerve gases, TNT, DNT, glucose, insulin, hCG-hormone, and drugs, including antibiotics or controlled substances such as cocaine.

Once the analyte 105 is selected in 110, in 120 directed evolution 122 may be performed to isolate nucleic acid enzymes, such as DNAzyme 124, RNAzyme 126, or aptazyme 125, which will catalyze ligation of substrate strands 133 and 135 in the presence of the analyte. The directed evolution 122 is preferably a type of in vitro selection method that selects molecules on the basis of their ability to interact with another constituent. Thus, the procedure of the directed evolution 122 may be selected to provide the nucleic acid enzymes that demonstrate enhanced ligation of the substrate fragments in the presence of the selected analyte 105 (thereby providing sensor sensitivity). The procedure also may be selected to exclude nucleic acid enzymes that demonstrate ligation in the presence of selected analytes, but additionally demonstrate ligation in the presence of non-selected analytes and/or other species present in the sample 102 (thereby providing sensor selectivity).

The directed evolution 122 may be any selection routine that provides nucleic acid enzymes that will catalyze the ligation of the substrate fragments in the presence of the desired analyte with the desired sensitivity and selectivity. Similarly, the directed evolution 122 may be utilized to identify aptamers that bind a selected analyte.

In one aspect, the directed evolution 122 may be initiated with a DNA library that includes a large collection of strands (e.g. 10¹⁶ sequence variants), each having a different variation of bases. Phosphoramidite chemistry may be utilized to generate the strands. The DNA library is then screened for strands that bind the analyte. These strands are isolated and amplified, such as by PCR. The amplified strands may then be mutated to reintroduce variation. These strands are then screened for strands that more effectively bind the analyte. By repeating the selection, amplification, and mutation sequence while increasing the amount of binding efficiency required for selection, strands that more effectively bind the analyte, thus providing greater sensitivity, may be generated.

In one aspect, a technique referred to as in vitro selection and evolution may be utilized to perform the directed evolution 122. Details regarding this technique may be found in Breaker, R., et al., “A DNA enzyme with Mg²⁺-dependent RNA phosphoesterase activity.,” Chem. Biol. 1995, 2:655-660; in Li, J., et al., “In Vitro Selection and Characterization of a Highly Efficient Zn(II)-dependent RNA-cleaving Deoxyribozyme.,” Nucleic Acids Res. 28,481-488 (2000); and in Cuenoud, B. et al., “A DNA Metalloenzyme with DNA Ligase Activity.,” Nature, 375,611-614 (1995).

In another aspect, nucleic acid enzymes having greater selectivity to a specific analyte may be obtained by introducing a negative selection process into the directed evolution 122. After selecting the strands having high sensitivity to the analyte, a similar selection, amplification, and mutation sequence may be applied, but to be selected, the strand must not bind closely related analytes.

For example, a DNAzyme may be selected that specifically binds Cu(II) and to a lesser extent Zn(II), while not significantly binding Mn(II), Ca(II), Co(II), or other competing metal ions. In one aspect, this may be achieved by isolating DNAzymes that bind Zn(II) and Cu(II), then removing any DNAzymes that bind Mn(II), Ca(II), or Co(II). In another aspect, DNAzymes that bind Mn(II), Ca(II), or Co(II) are first discarded and then those that bind Zn(II) and Cu(II) are isolated. In this manner, the selectivity of the DNAzyme may be increased. Details regarding one method to increase DNAzyme selectivity may be found in Bruesehoff, P. J., et al., “Improving Metal Ion Specificity During In Vitro Selection of Catalytic DNA,” Combinatorial Chemistry and High Throughput Screening, 5, 327-355 (2002).

DNA or RNA aptazymes may be obtained by known techniques including in vitro selection and rational design. An example of an in vitro selection process for cGMP or cAMP-dependent RNA-cleaving aptazymes may be found in Koizumi, M., et al., “Allosteric selection of ribozymes that respond to the second messengers cGMP and cAMP.” Nat. Struct. Biol., 6(11), 1062-71 (1999). An example of a rational design process for an ATP-dependent DNA-ligating aptazyme may be found in Levy, M., et al., “ATP-Dependent Allosteric DNA Enzymes.” Chem. Biol., 9, 417-26, (2002).

The DNA-RNAzymes 124, 126 and the DNA-RNA aptazyme 125 are nucleic acid enzymes having the ability to catalyze chemical reactions, such as ligation, in the presence of a co-factor. The DNAzyme 124 includes deoxyribonucleotides, while the RNAzyme 126 includes ribonucleotides. The DNA-RNA aptazyme 125 may include a DNA-RNAzyme modified with an aptamer motif to form an aptazyme requiring an aptamer target to catalyze ligation. The nucleotides from which the DNA-RNAzyme-aptazyme 124, 125, 126 are formed may be natural, unnatural, or modified nucleic acids. Peptide nucleic acids (PNAs), which include a polyamide backbone and nucleoside bases (available from Biosearch, Inc., Bedford, Mass., for example), also may be useful.

While DNAzymes, RNAzymes, and aptazymes derived from either can form duplexes with DNA-based substrate fragments, such as the substrate fragments 133 and 135 discussed below, the RNAzyme/Substrate duplex may be less stable than the DNAzyme/Substrate duplex. Additionally, DNAzymes and their aptazyme derivatives are easier to synthesize and more robust than their RNA-based counterparts.

The deoxyribonulceotides of the DNAzyme 124, DNA-based aptazymes and the complementary substrate strand fragments 133 and 135 may be substituted with their corresponding ribonucleotides, thus providing the RNAzyme 126, RNA-based aptazymes, and an RNA-based substrate fragments, respectively. For example, one or more ribo-cytosines may be substituted for the cytosines, one or more ribo-guanines may be substituted for the guanines, one or more ribo-adenosines may be substituted for the adenosines, and one or more uracils may be substituted for the thymines. In this manner, nucleic acid enzymes including DNA bases, RNA bases, or both may independently hybridize with complementary substrate strands that include DNA bases, RNA bases, or both.

After selecting an appropriate nucleic acid enzyme or enzymes in 120, a substrate 134 may be formed in a test sample 102 in 130. The substrate fragments 133 and 135 may be any oligonucleotides that may hybridize with and be ligated by the nucleic acid enzyme in the presence of the analyte 105. The oligonucleotides may be modified with any species capable of undergoing a ligation reaction in the presence of the nucleic acid enzyme. If the substrate 134 is released from the nucleic acid enzyme, the nucleic acid enzyme can react with additional substrate fragments 133, 135 to form additional substrates. In this manner, the nucleic acid enzyme may be catalytic.

To facilitate the release of the substrate 134 from the nucleic acid enzyme, one or more invasive DNA fragments may be added. In one aspect, an invasive DNA strand may be added that is partially complementary to the nucleic acid enzyme. For a more complete discussion of invasive DNA and how to tailor invasive DNA strands to facilitate the removal of a substrate from a nucleic acid enzyme, see Atny. Docket No. ELG05-051-US, filed Nov. 3, 2004, entitled “Nucleic Acid Enzyme Light-Up Sensor Utilizing Invasive DNA,” the portions addressing invasive DNA are incorporated herein by reference.

In one aspect, the resultant substrate 134 is complementary to and may hybridize with oligonucleotide functionalized particles 136. For example, if an oligonucleotide functionalized particle had a base sequence of 3′-TTCGTAGAGTTCG (SEQ ID NO. 6), an appropriate substrate fragment sequence for hybridization may be 5′-AAGCATCTCAAGC (SEQ ID NO. 3). In another aspect, if an oligonucleotide functionalized particle had a base sequence of 5′-CGGATAGTGTTCC (SEQ ID NO. 5), an appropriate substrate fragment sequence for hybridization may be 3′-GCCTATCACAAGG (SEQ ID NO. 4).

The particles 136 may be any species that demonstrate distance-dependent optical, electrical, or magnetic properties and are compatible with the operation of the sensor system. Suitable particles may include inorganic materials. In one aspect, the particles may include metals, such as gold, silver, copper, and platinum; semiconductors, such as CdSe, CdS, and CdS or CdSe coated with ZnS; and magnetic colloidal materials, such as those described in Josephson, Lee, et al., Angewandte Chemie, International Edition (2001), 40(17), 3204-3206. Specific useful particles may include ZnS, ZnO, TiO₂, Agl, AgBr, Hgl₂, PbS, PbSe, ZnTe, CdTe, In₂S₃, In₂Se₃, Cd₃P₂, Cd₃As₂, InAs, or GaAs. While gold nanoparticles are presently preferred, other fluorophores, such as dyes, inorganic crystals, quantum dots, and the like that undergo a distance-dependent color change also may be attached to oligonucleotides and utilized. For a more detailed treatment of how to prepare gold functionalized oligonucleotides, See U.S. Pat. No. 6,361,944; Mirkin, et al., Nature (London) 382, 607-09 (1996),; Storhoff, et al., J. Am. Chem. Soc., 20, 1959-64 (1998); and Storhoff, et al., Chem. Rev. (Washington, D.C.), 99, 1849-62 (1999).

In a preferred aspect, the particles are gold (Au) nanoparticles and have an average diameter from 5 to 100 nanometers (nm) or from 25 to 75 nm. In an aspect especially preferred at present, gold nanoparticles having an average diameter of from 45 to 55 nm are functionalized to the oligonucleotides. Because extinction coefficients increase with average particle diameter, larger particles are easier to observe with the naked eye at lower concentrations. Thus, larger particles can provide for increased sensitivity of the senor system in relation to smaller particles. For example, 50 nm particles may be observed in solution at concentrations of 0.1 nM, while smaller 13 nm particles are difficult to observe below a solution concentration of 0.5 nM.

In 140 the substrate 134 from 130 may be combined with the oligonucleotide functionalized particles 136. If the substrate 134 formed from the substrate fragments 133 and 135 in 130, an aggregate 132 may be formed as the particles 136 hybridize with the substrate 134. Thus, as the particles 136 are brought close together through hybridization with the substrate 134, the aggregate 132 forms. Considering the physical size of its components, the aggregate 132 may be quite large. In fact, transmission electron microscopy (TEM) studies suggest that individual aggregates may range from 100 nm to 1 micron, and may agglomerate to form larger structures.

Because the particles 136 demonstrate distance-dependent optical properties, the particles are one color when dispersed in the solution of the test sample 102 and undergo a color change when closely held in the aggregate 132 by the substrate 134. For example, when the particles 136 include gold nanoparticles, the resulting aqueous test sample turns from red to blue as the particles are brought close together by the substrate 134 to form the aggregate 132. The distance between the particles 136 upon hybridization to the substrate 134 may be selected by tailoring the length of the substrate fragments 133, 135.

In 150 the sample 102 is monitored for a color change. If a color change does not occur, the aggregate 132 was not formed and the analyte 105 is not present in the sample 102. If a color change does occur in 160, the aggregate 132 was formed and the analyte 105 is present in the sample 102. The color change signifies that the analyte 105 is an appropriate co-factor to catalyze ligation of the substrate fragments 133 and 135 into the substrate 134, which may then hybridize with the oligonucleotide functionalized particles 136. Thus, the analytic method 100 provides a “light-up” sensor system because a color change occurs in the presence of the analyte 105.

The degree the color changes in response to the analyte 105 may be quantified by colorimetric quantification methods known to those of ordinary skill in the art in 170. Various color comparator wheels, such as those available from Hach Co., Loveland, Colo. or LaMotte Co., Chestertown, Md. may be adapted for use with the present invention. Standard samples containing known amounts of the selected analyte may be analyzed in addition to the test sample to increase the accuracy of the comparison. If higher precision is desired, various types of spectrophotometers may be used to plot a Beer's curve in the desired concentration range. The color of the test sample may then be compared with the curve, and the concentration of the analyte present in the test sample determined. Suitable spectrophotometers include the Hewlett-Packard 8453 and the Bausch & Lomb Spec-20.

In yet another aspect, the method 100 may be modified to determine the sensitivity and selectivity of a ligase, such as a nucleic acid enzyme, for detecting the analyte 105. In this aspect, the substrate fragments 133, 135 are extended for at least 12 bases, so that the extension can hybridize with the particles 137, 139. Thus, the extended substrates and the particles 137, 139 may be combined with the analyte of interest 105, but without the DNA-RNAzymes-aptazymes 124, 126, 125 in 120. The ligase, such as one created by the directed evolution 122, may then be added. If the ligase joins the substrate fragments 133, 135 with the desired sensitivity and selectivity in the presence of the analyte 105 to form the substrate 134 and the associated aggregate 132, the ligase may be used to analyze for the analyte 105 in a colorimetric sensor system. In this aspect, the ligase or nucleic acid enzyme also may be considered an analyte. In this manner, multiple ligases generated from the directed evolution 122 may be tested for use in a colorimetric sensor system.

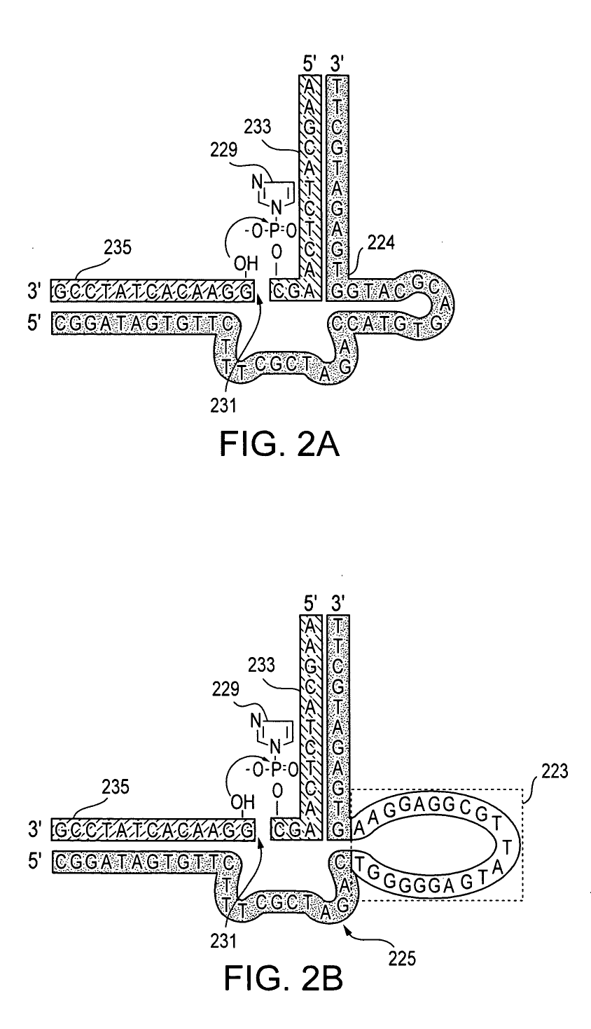

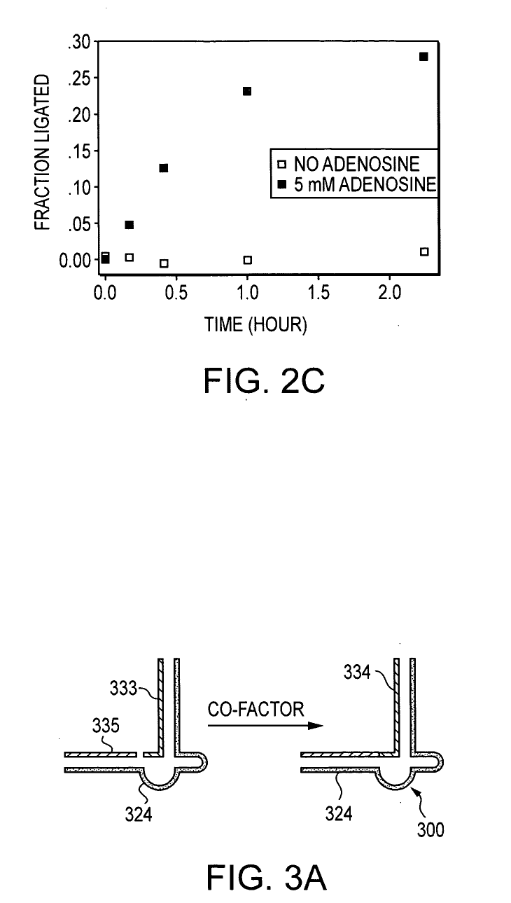

FIG. 2A represents a DNAzyme 224 that depends on Cu(II) and/or Zn(II) as a co-factor to display catalytic activity. The figure depicts what is believed to be the secondary structure of the DNAzyme 224. The synthesis and detailed description of the DNAzyme 224 may be found in Cuenoud, et al., “A DNA Metalloenzyme with DNA Ligase Activity,” Nature, 375, 611-14 (1995).

FIG. 2B represents a DNA aptazyme 225 that was formed from the DNAzyme 224 by replacing the hairpin structure of the DNAzyme 224 by an aptamer motif 223. Unlike the DNAzyme 224 of FIG. 2A, which may only require the Zn(II) and/or Cu(II) co-factor to catalyze ligation, the DNA aptazyme 225 of FIG. 2B may additionally require a specific aptamer target, such as adenosine, to catalyze ligation.

By providing an aptazyme sensor system with the required co-factor from the outset, the sensor system becomes responsive to analytes in the form of aptamer targets instead of co-factors. This behavior of the aptamer-modified DNA aptazyme 225 is shown in FIG. 2C, where in the presence of the Zn(II) co-factor, but in the absence of the adenosine target, ligation could not be detected. Thus, when provided with the appropriate co-factor, a DNA aptazyme catalyzes ligation in the presence of the aptamer target.

The DNA aptazyme 225 catalyzes ligation in the presence of a co-factor and adenosine by utilizing an aptamer motif that selectively binds adenosine. However, DNA aptazymes may be similarly designed relying on aptamer motifs that selectively bind other analytes. Table 1 below lists specific analytes, the aptamer motif or motifs that bind that analyte as a target, and the reference or references where each aptamer motif sequence is described. Any of these, and other, aptamers motifs may be adapted for use in the DNA aptazyme 125.

TABLE 1

| | | | |
|---|---|---|---|
| | | Aptamer Motif Sequence | |
| Analyte class | Example | (SEQ ID NO.) | Ref |

| | | | |
|---|---|---|---|
| Metal ions | K(I) | GGGTTAGGGTTAGGGTTAGGG | 1 |
| | | (SEQ ID NO. 7) | |
| | Zn(II) | AGGCGAGGUGAAAUGAGCGGUAAU | 2 |
| | | AGCCU | |
| | | (SEQ ID NO. 8) | |
| | Ni(II) | GGGAGAGGAUACUACACGUGAUAG | 3 |
| | | UCAGGGAACAUGACAAACACAGGG | |
| | | ACUUGCGAAAAUCAGUGUUUUGCC | |
| | | AUUGCAUGUAGCAG AAGCUUCCG | |
| | | (SEQ ID NO. 9) | |
| Organic dyes | Cibacron blue | GGGAGAATTCCCGCGGCAGAAGCCC | 4 |
| | | ACCTGGCTTTGAACTCTATGTTATTGG | |
| | | GTGGGGGAAACTTAAGAAAACTACC | |
| | | ACCCTTCAACATTACCGCCCTTCAGCC | |
| | | TGCCAGCGCCCTGCAGCCCGGGAAG | |
| | | CTT | |
| | | (SEQ ID NO. 10) | |
| | Malachite green | GGAUCCCGACUGGCGAGAGCCAGG | 5 |
| | | UAACGA AUGGAUCC | |
| | | (SEQ ID NO. 11) | |
| | Sulforhodamine B | CCGGCCAAGGGTGGGAGGGAGGGG | 6 |
| | | GCCGG | |
| | | (SEQ ID NO. 12) | |
| Small organic | Biotin | AUGGCACCGACCAUAGGCUCGGGU | 7 |
| molecules | | UGCCAGAGGUUCCACACUUUCAUC | |
| | | GAAAAGCCUAUGC | |
| | | (SEQ ID NO. 13) | |
| | Theophylline | GGCGAUACCAGCCGAAAGGCCCUU | 8 |
| | | GGCAGCGUC | |
| | | (SEQ ID NO. 14) | |
| | Adenine | GAUAGGACGAUUAUCGAAAAUCAC | 9 |
| | | CAGAUUGGACCCUGGUUAACGAUC | |
| | | CAUU | |
| | | (SEQ ID NO. 15) | |
| | Cocaine | GACAAGGAAAATCCTTCAATGAAGTG | 10 |
| | | GGTC | |
| | | (SEQ ID NO. 16) | |
| | Dopamine | GGGAAUUCCGCGUGUGCGCCGCG | 11 |
| | | GAAGAGGGAAUAUAGAGGCCAGCA | |
| | | CAUAGUGAGGCCCUCCUCCC | |
| | | (SEQ ID NO. 17) | |
| Amino acids | Arginine | GGGAGCUCAGAAUAAACGCUCAAG | 12 |
| | | GAGGACCGUGCACUCCUCGAACAU | |
| | | UUCGAGAUGAGACACGGAUCCUGC | |
| | | (SEQ ID NO. 18) | |
| | Citrulline | GACGAGAAGGAGUGCUGGUUAUAC | 13 |
| | | UAGCGGUUAGGUCACUCGUC | |
| | | (SEQ ID NO. 19) | |
| Nucleosides & | ATP | ACCTGGGGGAGTATTGCGGAGGAAG | 14 |
| nucleotides | | GT | |
| | | (SEQ ID NO. 20) | |
| | cAMP | GGAAGAGAUGGCGACUAAAACGAC | 15 |
| | | UUGUCGC | |
| | | (SEQ ID NO. 21) | |
| | GTP | UCUAGCAGUUCAGGUAACCACGUA | 16 |
| | | AGAUACGGGUCUAGA | |
| | | (SEQ ID NO. 22) | |
| | Guanosine | GGGAGCUCAGAAUAAACGCUCAAC | 17 |
| | | CCGACAGAUCGGCAACGCCNUGUU | |
| | | UUCGACANGAGACACCGAUCCUGC | |
| | | ACCAAAGCUUCC | |
| | | (SEQ ID NO. 23) | |
| RNA | TAR-RNA | GCAGTCTCGTCGACACCCAGCAGCG | 18 |
| | | CATGTAACTCCCATACATGTGTGTGCT | |
| | | GGATCCGACGCAG | |
| | | (SEQ ID NO. 24) | |
| Biological | CoA | GGGCACGAGCGAAGGGCAUAAGCU | 19 |
| cofactors | | GACGAAAGUCAGACAAGACAUGGU | |
| | | GCCC | |
| | | (SEQ ID NO. 25) | |
| | NMN | GGAACCCAACUAGGCGUUUGAGGG | 20 |
| | | GAUUCGGCCACGGUAACAACCCCU | |
| | | C | |
| | | (SEQ ID NO. 26) | |
| | FAD | GGGCAUAAGGUAUUUAAUUCCAUA | 21 |
| | | CAAGUUUACAAGAAAGAUGCA | |
| | | (SEQ ID NO. 27) | |
| | Porphyrin | TAAACTAAATGTGGAGGGTGGGACG | 22 |
| | | GGAAGAAGTTTA | |
| | | (SEQ ID NO. 28) | |
| | Vitamin B12 | CCGGUGCGCAUAACCACCUCAGUG | 23 |
| | | CGAGCAA | |
| | | (SEQ ID NO. 29) | |
| Amino- | Tobramycin | GGGAGAAUUCCGACCAGAAGCUUU | 24 |
| glycosides | | GGUUGUCUUGUACGUUCACUGUU | |
| | | ACGAUUGUGUUAGGUUUAACUACA | |
| | | CUUUGCAAUCGCAUAUGUGCGUCU | |
| | | ACAUGGAUCCUCA | |
| | | (SEQ ID NO. 30) | |
| Oligo- | Cellobiose | GCGGGGTTGGGCGGGTGGGTTCGC | 25 |
| saccharides | | TGGGCAGGGGGCGAGTC | |
| | | (SEQ ID NO. 31) | |
| Poly- | Sephadex | UACAGAAUGGGUUGGUAGGCAUAC | 26 |
| saccharides | | CUAAUCGAGAAUGAUA | |
| | | (SEQ ID NO. 32) | |
| Antibiotics | Viomycin | GGAGCUCAGCCUUCACUGCAAUGG | 27 |
| | | GCCGCUAGGUUGAUGUGCAGUGA | |
| | | AGUCAGCUGAGGCCCAGGGCUGAA | |
| | | AGGAUCGCCCUCCUCGACUCGUGG | |
| | | CACCACGGUCGGAUCCAC | |
| | | (SEQ ID NO. 33) | |
| | Streptomycin | GGAUCGCAUUUGGACUUCUGCCCA | 28 |
| | | GGGGGCACCACGGUCGGAUCC | |
| | | (SEQ ID NO. 34) | |
| | Tetracycline | GGCCUAAAACAUACCAGAUUUCGA | 29 |
| | | UCUGGAGAGGUGAAGAAUUCGACC | |
| | | ACCUAGGCCGGU | |
| | | (SEQ ID NO. 35) | |
| | Vasopressin | ACGTGAATGATAGACGTATGTCGAGT | 30 |
| | | TGCTGTGTGCGGATGAACGT | |
| | | (SEQ ID NO. 36) | |
| Peptides | Substance P | GGGAGCUGAGAAUAAACGCUCAAG | 31 |
| | | GGCAACGCGGGCACCCCGACAGGU | |
| | | CCAAAAACGCACCGACGCCCGGCCG | |
| | | AAGAAGGGGAUUCGACAUGAGGCC | |
| | | CGGAUCCGGC | |
| | | (SEQ ID NO. 37) | |
| Enzymes | HIV | UCCGUUUUCAGUCGGGAAAAACUG | 32 |
| | Rev Transcriptase | (SEQ ID NO. 38) | |
| | Human thrombin | GGTTGGTGTGGTTGG | 33 |
| | | (SEQ ID NO. 39) | |
| Growth | VEGF₁₆₅ | GCGGUAGGAAGAAUUGGAAGCGC | 34 |
| factors | | (SEQ ID NO. 40) | |
| Transcription | NF-κB | GGGAUAUCCUCGAGACAUAAGAAA | 35 |
| factors | | CAAGAUAGAUCCUGAAACUGUUUU | |
| | | AAGGUUGGCCGAUCUUCUGCUCGA | |
| | | GAAUGCAUGAAGCGUUCCAUAUUU | |
| | | UU | |
| | | (SEQ ID NO. 41) | |
| Antibodies | Human IgE | GGGGCACGTTTATCCGTCCCTCCTAG | 36 |
| | | TGGCGTGCCCC | |
| | | (SEQ ID NO. 42) | |
| Gene | Elongation | GGGGCUAUUGUGACUCAGCGGUU | 37 |
| Regulatory | factor Tu | CGACCCCGCUUAGCUCCACCA | |
| factors | | (SEQ ID NO. 43) | |
| Cell adhesion | Human CD4 | UGACGUCCUUAGAAUUGCGCAUUC | 38 |
| molecules | | CUCACACAGGAUCUU | |
| | | (SEQ ID NO. 44) | |
| cells | YPEN-1 | ATACCAGCTTATTCAATTAGGCGGTG | 39 |
| | endothelial | CATTGTGGTTGGTAGTATACATGAGG | |
| | | TTTGGTTGAGACTAGTCGCAAGATAT | |
| | | AGATAGTAAGTGCAATCT | |
| | | (SEQ ID NO. 45) | |
| Viral/bacterial | Anthrax spores | Sequences are not given | 40 |
| components | Rous | AGGACCCUCGAGGGAGGUUGCGCA | 41 |
| | sarcoma virus | GGGU | |
| | | (SEQ ID NO. 46) | |

Reference Listing for Table 1
1 Ueyama, H., Takagi, M. & Takenaka, S. A novel potassium sensing in aqueous media with a synthetic oligonucleotide derivative. fluorescence resonance energy transfer associated with guanine quartet-potassium ion complex formation. J. Am. Chem. Soc. 124, 14286-14287 (2002).
2 Ciesiolka, J. & Yarus, M. Small RNA-divalent domains. RNA 2, 785-793 (1996)
3 Hofmann, H. P., Limmer, S., Hornung, V. & Sprinzl, M. Ni2+-binding RNA motifs with an asymmetric purine-rich internal loop and a G-A base pair. RNA 3, 1289-300. (1997).
4 Ellington, A. D. & Szostak, J. W. In vitro selection of RNA molecules that bind specific ligands. Nature (London) 346, 818-22 (1990).
5 Grate, D. & Wilson, C. Laser-mediated, site-specific inactivation of RNA transcripts. Proc. Natl. Acad. Sci. U.S.A. 96, 6131-6136 (1999).
6 Wilson, C. & Szostak, J. W. Isolation of a fluorophore-specific DNA aptamer with weak redox activity. Chemistry & Biology 5, 609-617 (1998).
7 Wilson, C., Nix, J. & Szostak, J. Functional Requirements for Specific Ligand Recognition by a Biotin-Binding RNA Pseudoknot. Biochemistry 37, 14410-14419 (1998).
8 Zimmermann, G. R., Wick, C. L., Shields, T. P., Jenison, R. D. & Pardi, A. Molecular interactions and metal binding in the theophylline-binding core of an RNA aptamer. Rna 6, 659-667 (2000).
9 Meli, M., Vergne, J., Decout, J.-L. & Maurel, M.-C. Adenine-aptamer complexes. A bipartite RNA site that binds the adenine nucleic base. J. Biol. Chem. 277, 2104-2111 (2002).
10 Stojanovic, M. N., de Prada, P. & Landry, D. W. Fluorescent Sensors Based on Aptamer Self-Assembly. J. Am. Chem. Soc. 122, 11547-11548 (2000).
11 Mannironi, C., Di Nardo, A., Fruscoloni, P. & Tocchini-Valentini, G. P. In vitro selection of dopamine RNA ligands. Biochemistry 36, 9726-9734 (1997).
12 Connell, G. J., Illangesekare, M. & Yarus, M. Three small ribooligonucleotides with specific arginine sites. Biochemistry 32, 5497-502 (1993).
13 Famulok, M. Molecular Recognition of Amino Acids by RNA-Aptamers: An L-Citrulline Binding RNA Motif and Its Evolution into an L-Arginine Binder. J. Am. Chem. Soc. 116, 1698-706 (1994).
14 Sassanfar, M. & Szostak, J. W. An RNA motif that binds ATP. Nature (London) 364, 550-3 (1993).
15 Koizumi, M. & Breaker, R. R. Molecular Recognition of cAMP by an RNA Aptamer. Biochemistry 39, 8983-8992 (2000).
16 Davis, J. H. & Szostak, J. W. Isolation of high-affinity GTP aptamers from partially structured RNA libraries. Proc. Natl. Acad. Sci. U.S.A. 99, 11616-11621 (2002).
17 Connell, G. J. & Yarus, M. RNAs with dual specificity and dual RNAs with similar specificity. Science (Washington, D. C.) 264, 1137-41 (1994).
18 Boiziau, C., Dausse, E., Yurchenko, L. & Toulme, J.-J. DNA aptamers selected against the HIV-1 trans-activation-responsive RNA element form RNA-DNA kissing complexes. J. Biol. Chem. 274, 12730-12737 (1999).
19 Burke, D. & Hoffman, D. A Novel Acidophilic RNA Motif That Recognizes Coenzyme A. Biochemistry 37, 4653-4663 (1998).
20 Lauhon, C. T. & Szostak, J. W. RNA aptamers that bind flavin and nicotinamide redox cofactors. J. Am. Chem. Soc. 117, 1246-57 (1995).
21 Roychowdhury-Saha, M., Lato, S. M., Shank, E. D. & Burke, D. H. Flavin Recognition by an RNA Aptamer Targeted toward FAD. Biochemistry 41, 2492-2499 (2002).
22 Chinnapen, D. J. F. & Sen, D. Hemin-Stimulated Docking of Cytochrome c to a Hemin-DNA Aptamer Complex. Biochemistry 41, 5202-5212 (2002).
23 Lorsch, J. R. & Szostak, J. W. In vitro selection of RNA aptamers specific for cyanocobalamin. Biochemistry 33, 973-82 (1994).
24 Wang, Y., Killian, J., Hamasaki, K. & Rando, R. R. RNA Molecules That Specifically and Stoichiometrically Bind Aminoglycoside Antibiotics with High Affinities. Biochemistry 35, 12338-12346 (1996).
25 Yang, Q., Goldstein, I. J., Mei, H.-Y. & Engelke, D. R. DNA ligands that bind tightly and selectively to cellobiose. Proc. Natl. Acad. Sci. U.S.A. 95, 5462-5467 (1998).
26 Srisawat, C., Goldstein, I. J. & Engelke, D. R. Sephadex-binding RNA ligands: rapid affinity purification of RNA from complex RNA mixtures. Nucleic Acids Res. 29, E4/1-E4/5 (2001).
27 Wallis, M. G. et al. In vitro selection of a viomycin-binding RNA pseudoknot. Chem. Biol. 4, 357-366 (1997).
28 Wallace, S. T. & Schroeder, R. In vitro selection and characterization of streptomycin-binding RNAs: recognition discrimination between antibiotics. Rna 4, 112-123 (1998).
29 Berens, C., Thain, A. & Schroeder, R. A tetracycline-binding RNA aptamer. Bioorganic & Medicinal Chemistry 9, 2549-2556 (2001).
30 Williams, K. P. et al. Bioactive and nuclease-resistant L-DNA ligand of vasopressin. Proc. Natl. Acad. Sci. U.S.A. 94, 11285-11290 (1997).
31 Nieuwlandt, D., Wecker, M. & Gold, L. In Vitro Selection of RNA Ligands to Substance P. Biochemistry 34, 5651-9 (1995).
32 Tuerk, C., MacDougal, S. & Gold, L. RNA pseudoknots that inhibit human immunodeficiency virus type 1 reverse transcriptase. Proc. Natl. Acad. Sci. U.S.A. 89, 6988-92 (1992).
33 Bock, L. C., Griffin, L. C., Latham, J. A., Vermaas, E. H. & Toole, J. J. Selection of single-stranded DNA molecules that bind and inhibit human thrombin. Nature (London) 355, 564-6 (1992).
34 Ruckman, J. et al. 2′-Fluoropyrimidine RNA-based aptamers to the 165-amino acid form of vascular endothelial growth factor (VEGF165). Inhibition of receptor binding and VEGF-induced vascular permeability through interactions requiring the exon 7-encoded domain. J. Biol. Chem. 273, 20556-20567 (1998).
35 Lebruska, L. L. & Maher, L. J., III. Selection and Characterization of an RNA Decoy for Transcription Factor NF-kB. Biochemistry 38, 3168-3174 (1999).
36 Wiegand, T. W. et al. High-affinity oligonucleotide ligands to human IgE inhibit binding to Fc epsilon receptor I.J. Immunol. 157, 221-30 (1996).
37 Nazarenko, I. A. & Uhlenbeck, O. C. Defining a Smaller RNA Substrate for Elongation Factor Tu. Biochemistry 34, 2545-52 (1995).
38 Davis, K. A., Lin, Y., Abrams, B. & Jayasena, S. D. Staining of cell surface human CD4 with 2′-F-pyrimidine-containing RNA aptamers for flow cytometry. Nucleic Acids Res. 26, 3915-3924 (1998).
39 Blank, M., Weinschenk, T., Priemer, M. & Schluesener, H. Systematic evolution of a DNA aptamer binding to rat brain tumor microvessels. Selective targeting of endothelial regulatory protein pigpen. J. Biol. Chem. 276, 16464-16468 (2001).
40 Bruno, J. G. & Kiel, J. L. In vitro selection of DNA aptamers to anthrax spores with electrochemiluminescence detection. Biosensors & Bioelectronics 14, 457-464 (1999).
41 Pan, W. et al. Isolation of virus-neutralizing RNAs from a large pool of random sequences. Proc. Natl. Acad. Sci. U.S.A. 92, 11509-13 (1995).

As may be seen from the base pairs in FIGS. 2A and 2B, the DNAzyme 224 or its adenosine-dependent aptazyme 225 may hybridize with complementary substrate fragments 233 and 235 that include an imidazole modified phosphate 229 and a hydroxyl group. The depicted substrate fragments 233 and 235 are formed from doexyribonucleosides. While one base sequence is shown for the DNAzyme (FIG. 2A), the DNA-aptazyme (FIG. 2B), and the substrate strands 233, 235, the bases may be changed on the enzyme and substrate strands to maintain the pairings. For example, any C on either strand may be changed to T, as long as the paired base is changed from G to A.

The base pairing regions of the DNAzyme-aptazyme 224, 225 and the complementary substrate fragments 233 and 235 may be extended or truncated, as long as sufficient bases exist to maintain the desired ligation of the substrate fragments. While many modifications to the enzyme and substrate are possible, modifications made to the catalytic core region of the enzyme can have significant effects on the catalytic efficiency or analyte specificity of the enzyme. A more detailed discussion of such modifications and the resulting effects on catalytic activity may be found in Brown, A., et al., “A Lead-dependent DNAzyme with a Two-Step Mechanism,” Biochemistry, 42, 7152-61 (2003).

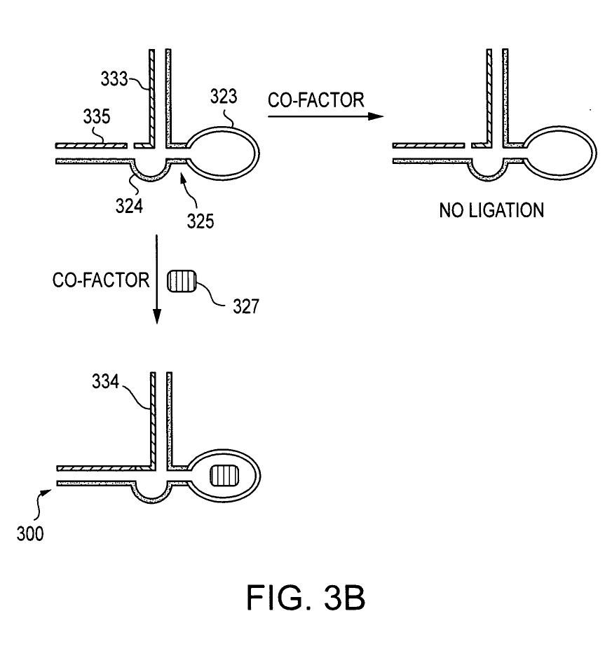

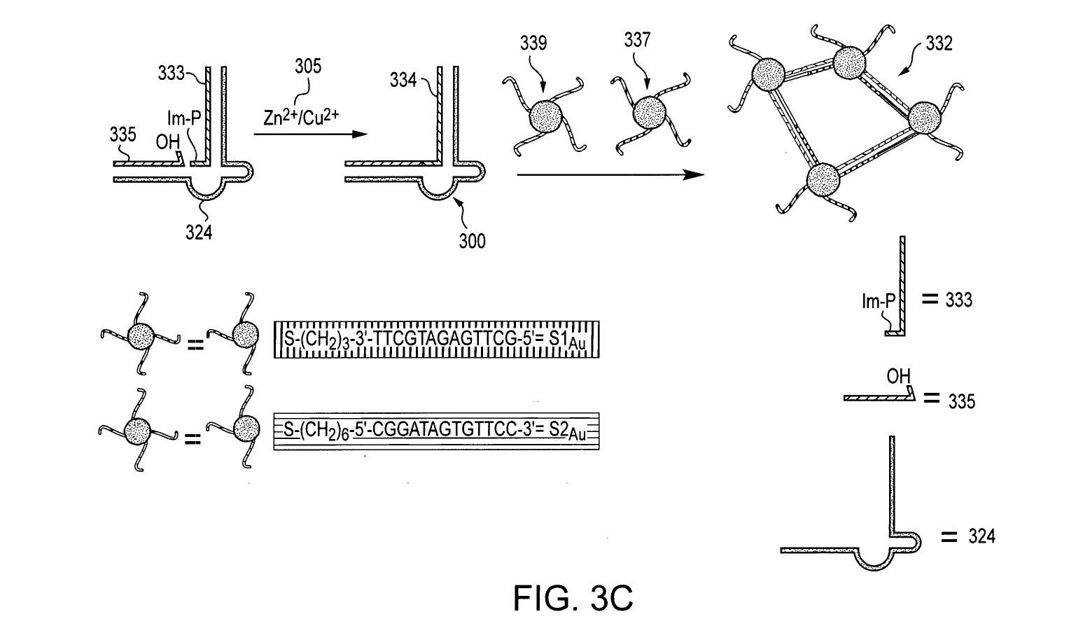

Between the imidazole-modified phosphate 229 and the hydroxyl group lies the ligation site 231, where the DNAzyme-aptazyme 224, 225 may ligate the substrate fragments 233 and 235 to form the substrate 234. This ligation reaction results in the ligation of the 3′-end of the substrate fragment 233 with the 5′-end of the substrate fragment 235 as discussed further with regard to FIG. 3.

In one aspect, the substrate fragment 233 may be modified or activated with an imidazole, while the substrate fragment 235 includes a hydroxyl (—OH) group. In another aspect, the substrate fragment 233 may be phosphorylated and then activated with the imidazole. In this aspect, it is believed that the DNAzyme-aptazyme 224, 225 induces ligation of the substrate fragments 233, 235 by catalyzing nucleophilic attack of the phosphor center on 233 by the hydroxyl group on 235 to form a phosphodiester bond with the imidazole functioning as the leaving group.

The DNAzyme-aptazyme 224, 225 and the complementary substrate fragments 233 and 235 all may be separate strands, as depicted in FIGS. 2A and 2B, or the DNAzyme-aptazyme and one or both of the substrate fragments may be part of the same nucleic acid strand. When the DNAzyme-aptazyme and the complementary substrate fragments are different nucleic acid strands, the DNAzyme-aptazyme may be referred to as a “trans-acting enzyme.” Trans-acting enzymes have the advantage of being able to join multiple complementary substrate fragments, thus being catalytic. If the DNAzyme-aptazyme and the complementary substrate fragments are part of the same nucleic acid strand, the DNAzyme-aptazyme may be referred to as a “cis-acting enzyme.”

FIG. 3A represents the ligation of substrate fragments 333 and 335 to form substrate 334 by DNAzyme 324. The ligation results in the formation of a ligation product 300 that includes the substrate 334 hybridized with the DNAzyme 324 in the presence of the co-factor. The substrate 334 may then leave the ligation product 300 to hybridize with oligonucleotide functionalized particles 337 and 339, as described further with regard to FIG. 3C. In this manner the DNAzyme 324 can participate in additional ligation reactions between additional complementary substrate fragments.

FIG. 3B represents the ligation of the substrate fragments 333 and 335 to form the substrate 334 by DNA aptazyme 325. The ligation results in the formation of the hybridized ligation product 300 in the presence of a metal co-factor and the aptamer target 327. Without the appropriate target 327 for the aptamer 323, the DNA aptazyme 325 does not ligate the substrate fragments 333 and 335 into the substrate 334. Thus, unlike in FIG. 3A where the co-factor was the analyte, in FIG. 3B the aptamer target 327 serves as the analyte.

FIG. 3C represents the formation of the substrate 334 from the substrate fragments 333 and 335 in the presence of the Zn(II) and/or Cu(II) analytes 305 by the DNAzyme 324. The DNAzyme 324 may be referred to as “E47,” which exhibits high activity in the presence of the Zn(II) cation and a lesser activity in the presence of the Cu(II) cation. The aggregate 332 then may be formed as the substrate 334 hybridizes to 3′ and 5′ thiol-oligonucleotide functionalized particles 337 and 339, respectively. In one aspect, the hybridization of the particles 337, 339 with the substrate 334 may assist in separating the substrate from the DNAzyme 324. In another aspect, when the E47 DNAzyme is utilized, invasive DNA (not shown) that is complementary from the 10^{th} base of the 5′-end to the 38^{th} base of the E47 strand may be added to speed the separation of the substrate strand 334 from the DNAzyme 324.

As the oligonucleotide functionalized particles 337 and 339 hybridize with the substrate 334, the color of the solution changes from red to blue. Formation of the blue aggregate 332 in the presence of the analyte 305 adds blue color to the red solution as the particles are brought closer together in the aggregate 332, thus giving a purple solution. If enough of the analyte 305 is present in the sample, enough of the substrate 334 will form to bind substantially all of the particles together into aggregates, thus providing a blue solution due to the close proximity of the nanoparticles.

The alignment of the particles (tail-to-tail or head-to-tail) with respect to each other may influence how tightly the moieties that form the aggregate bind together. FIG. 3C depicts that the aggregate 332 may be formed from the substrate 344 and the functionalized particles 337, 339 where the particles hybridize in a tail-to-tail arrangement with the substrate. Tail-to-tail or head-to-tail hybridization may be selected by reversing the end of the oligonucleotide to which the particle is attached. At present, the tail-to-tail hybridization arrangement of FIG. 3C is preferred because head-to-tail hybridization may produce less stable aggregates. However, this steric hindrance may be reduced through a reduction in the average diameter of the particles or through the use of a longer substrate, for example.

The sensor system, including the substrate fragments and the oligonucleotide functionalized particles may be provided in the form of a kit. In one aspect, the kit includes the desired analyte specific ligase that is at least partially complementary to the substrate fragments. In yet another aspect, the kit excludes the ligase, which is then provided by the user or provided separately. In this aspect, the kit also may be used to determine the specificity and/or selectivity of various ligases to a selected analyte. Thus, the kit may be used to select an appropriate ligase in addition to detecting the analyte. In yet another aspect, the kit includes an exterior package that encloses a DNAzyme-aptazyme, complementary substrate fragments, and oligonucleotide functionalized particles. In yet another aspect, the kit includes invasive DNA.

One or more of these components may be separated into individual containers, or the DNAzyme-aptazyme may be provided hybridized to the substrate fragments. If separated, the substrate fragments may be hybridized to the DNAzyme or aptazyme before introducing the sample. The invasive DNA may be held in a separate container so it may be added to the sample before, during, or after combination with the particles. Additional buffers and/or pH modifiers may be provided in the kit to adjust the ionic strength and/or pH of the sample.

The containers may take the form of bottles, tubs, sachets, envelopes, tubes, ampoules, and the like, which may be formed in part or in whole from plastic, glass, paper, foil, MYLAR®, wax, and the like. The containers may be equipped with fully or partially detachable lids that may initially be part of the containers or may be affixed to the containers by mechanical, adhesive, or other means. The containers also may be equipped with stoppers, allowing access to the contents by syringe needle. In one aspect, the exterior package may be made of paper or plastic, while the containers are glass ampoules.

The exterior package may include instructions regarding the use of the components. Color comparators; standard analyte solutions, such as a 10 μm solution of the analyte; and visualization aids, such as thin layer chromatography (TLC) plates, test tubes, and cuvettes, also may be included. Containers having two or more compartments separated by a membrane that may be removed to allow mixing may be included. The exterior package also may include filters and reagents that allow preparation of the sample for analysis. Suitable reagents for preparing the sample may include dilution reagents, pH modification reagents, ionic strength modification reagents, and the like.

In another aspect, in addition to the sensor system of the present invention, the kit also may include multiple sensor systems to further increase the reliability of analyte determination and reduce the probability of user error. In one aspect, multiple light-up sensor systems in accord with the present invention may be included. In another aspect, a “light-down” sensor system may be included with the light-up sensor system of the present invention.

The presently claimed sensor system may be considered a light-up sensor because a color change occurs (red to blue) in the presence of the analyte. Conversely, in a light-down sensor system, a color change is not observed in the presence of the analyte. By virtually eliminating the background ligation, which could otherwise provide a false result by lighting up when the analyte is absent, the present light-up system provides a useful increase in the accuracy of analyte detection and quantification. Combining a sensor system using light-down chemistry with the presently claimed light-up sensor may reduce the probability of an inaccurate analyte determination.

Suitable light-down sensors for inclusion in the presently claimed kit may rely on DNAzyme/Substrate/particle aggregates that are not formed in the presence of the selected analyte. Thus, for these sensors, a color change from aggregate formation is observed when the selected analyte is not present in the sample. In one aspect, these light-down sensors may rely on a tail-to-tail particle arrangement coupled with nanoparticles having average diameters of about 43 nm to provide aggregation at room temperature in the absence of the analyte. A more detailed description of suitable light-down sensor systems for inclusion in the presently claimed kit may be found, for example, in U.S. patent application Ser. No. 10/756,825, filed Jan. 13, 2004, entitled “Biosensors Based on Directed Assembly of Particles,” which is hereby incorporated by reference.

The preceding description is not intended to limit the scope of the invention to the preferred embodiments described, but rather to enable a person of ordinary skill in the art to make and use the invention. Similarly, the examples below are not to be construed as limiting the scope of the appended claims or their equivalents, and are provided solely for illustration. It is to be understood that numerous variations can be made to the procedures below, which lie within the scope of the appended claims and their equivalents.

### EXAMPLES

DNA samples were purchased from Integrated DNA Technologies Inc., Coralville, Iowa. The substrates and enzyme portions of the DNAzyme were purified by HPLC prior to use. Additional materials, such as adenosine and ZnCl₂ were purchased from Aldrich, Milwaukee, Wis. and used as received.

The sequences obtained from Integrated DNA Technologies include the E47 DNAzyme 224 (SEQ ID NO: 1) and the E47 DNAzyme modified into an adenosine-dependent aptazyme E47+Ade 225 (SEQ ID NO: 2) along with the substrate fragments 233 (SEQ ID NO: 3) and 235 (SEQ ID NO: 4). Each of these strands are given in Table 2 below and shown in FIG. 2. The sequences of the 5′DNA_{Au} 339 (SEQ ID NO: 5) and the 3′DNA_{Au} 337 (SEQ ID NO: 6) oligonucleotide functionalized gold nanoparticles from FIG. 3C that hybridize these substrate fragments also are listed.

TABLE 2

| | | SEQ |
|---|---|---|
| Name | Sequences | ID |
| E47 | CGGATAGTGTTCTTTCGCTAGAC | 1 |
| | CATGTGACGCATGGTGAGATGCT | |
| | T | |
| E47 + Ade | CGGATAGTGTTCTTTCGCTAGAC | 2 |
| | TGGGGGAGTATTGCGGAGGAAGT | |
| | GAGATGCTT | |
| SF233 | AAGCATCTCAAGC | 3 |
| SF235 | GGAACACTATCCG | 4 |
| 5′DNA_{AU} | CGGATAGTGTTCC | 5 |
| 3′DNA_{AU} | GCTTGAGATGCTT | 6 |

All sequences are listed from 5′ to 3′. For 5′DNA_{AU} and 3′DNA_{AU}, a gold nanoparticle was attached via a 5′- and 3′-end thiol-linkage, respectively. For SF233, the 3′-end was phosphorylated

### Example 1

### Formation Gold Nanoparticles

Gold nanoparticles having an average diameter of 50 nm were prepared by the citrate reduction method. Two-hundred milliliters of a 0.3 mM HAuCl₄ solution was heated to reflux while stirring. To this solution was added 1.8 mL of a 38.8 mM sodium citrate solution. When the color of the resultant solution changed to red, the solution was refluxed for another 30 minutes and then allowed to cool to room temperature. The solution was then filtered trough a glass frit and the nanoparticles were collected.

### Example 2

### Oligonucleotide Functionalization of Gold Nanoparticles

3′- and 5′-thiol-modified DNA was activated by incubating with Tri(2-carboxyethyl)phosphine hydrochloride (TCEP). Typically, 20 μL of 1 mM DNA was incubated with 2 μL of 20 mM TCEP at room temperature for 1 hour. The mixture was then directly added into 6 mL of the gold nanoparticles from Example 1. After incubation for 16 hours at room temperature, 0.6 mL of buffer containing 1 M of NaCl and 100 mM of Tris acetate (pH 8.2) was dropwise added to the stirred nanoparticle solution. After incubation for another day, the nanoparticles were centrifuged at 9000 rpm for 10 minutes. The supernatant was removed and nanoparticles were re-dispersed in buffer containing 100 mM NaCl, 25 mM tris acetate (pH 8.2). This centrifugation process was repeated 3 times to remove the free DNA from the solution. The average diameter of the gold nanoparticles was verified by transmission electronic microscope (JEOL 2010).

### Example 3

### Imidazole Modification of Substrate Fragment

A substrate fragment with a phosphorylated 3′-end, such as 233 from FIG. 2, was purchased from Integrated DNA Technologies Inc. The imidazole group was attached to the 3′-phosphate group by reacting 20 μL of a 100 μM solution containing the phosphorylated fragment with 2.5 μL of 1 M imidazole (pH 6.0, adjusted with concentrated HCl) and 2.5 μL of 1.5 M EDC.HCl at room temperature for 1 hour. The resultant mixture was desalted with a PD-10 (Amersham Biosciences) desalting column, and the fraction of 0.5 to 1.5 mL was collected. The DNA concentration of the eluted fraction was determined by monitoring the absorbance at 260 nm.

### Example 4

### Preparation of a DNAzyme Sensor System

The imidazole-activated substrate fragment from Example 3 was dissolved in 300 mM KCl, 20 mM MgCl₂, 30 mM HEPES buffer (pH 7.0) with a second substrate fragment, such as 235 from FIG. 2A, and a nucleic acid enzyme, such as the DNAzyme 224 from FIG. 2A. After about 30 minutes at room temperature, the ligation reaction was initiated by adding 1 μL of 50× concentrated Cu²⁺ or Zn²⁺ solution. After 30 minutes, 5 μL of the solution was transferred to another tube containing 45 μL of 0.09 nM DNA-functionalized gold nanoparticles (mixture of 3′- and 5′-thiol-functionalized DNA with equal concentration), 300 mM NaCl, 30 mM tris acetate buffer (pH 8.2) and 1 μM of 29anti47E DNA (5′-ACC ATG CGT CAC ATG GTC TAG CGA AAG AA-3′) (SEQ ID NO: 42). Shortly after mixing, the tube containing the nanoparticles was placed in a beaker containing 10 mL of boiling water and was allowed to cool to room temperature for 20 minutes. The UV-vis extinction spectra of the samples were then collected or the nanoparticles were spotted onto an alumina TLC plate for visualization.

### Example 5

### Confirmation of Ligation Product Formation

The formation of the substrate from the substrate fragments was confirmed by monitoring the kinetics of both Cu(II) and Zn(II) induced ligation. The 3′-end of a substrate fragment, such as the fragment 235 from FIG. 2A, was labeled with a FAM (6′-carboxylfluorescein) fluorescent dye. The labeled substrate fragments that were ligated into a substrate were separated from the labeled substrate fragments that were not ligated into a substrate by polyacrylamide gel electrophoresis.

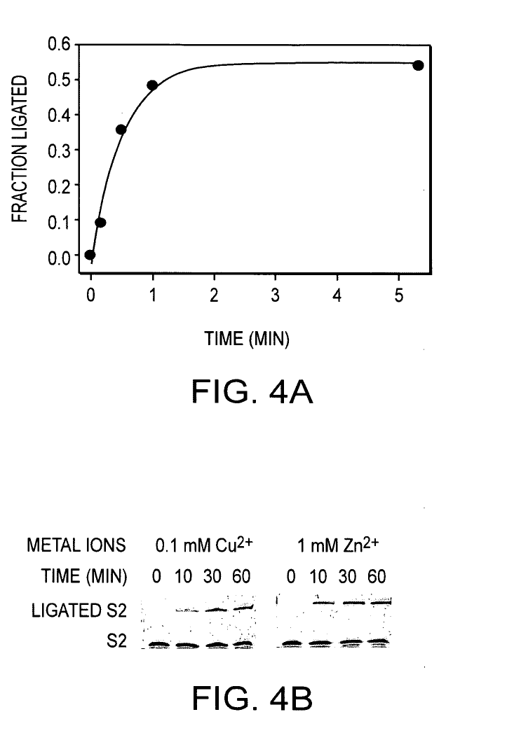

As shown in FIG. 4A, the fraction of the labeled substrate fragments that were ligated increased with time for both Cu(II) and Zn(II). The ligation kinetics parameters were obtained by quantifying the intensity of the gel bands depicted in FIG. 4B. For example, in HEPES buffer at pH 7.0, the ligation rate was 2 hour⁻¹ in the presence of 1 mM ZnCl₂. The images were obtained with a fluorescence image analysis system (FLA-3000G, Fuji) with 473 nm laser excitation. The filter was set to 520 nm. The kinetics data was fit to the equation y=y₀+a(1−e^{-kt}), where k is the observed rate constant.

### Example 6

### Confirmation of Nanoparticle Aggregate Formation

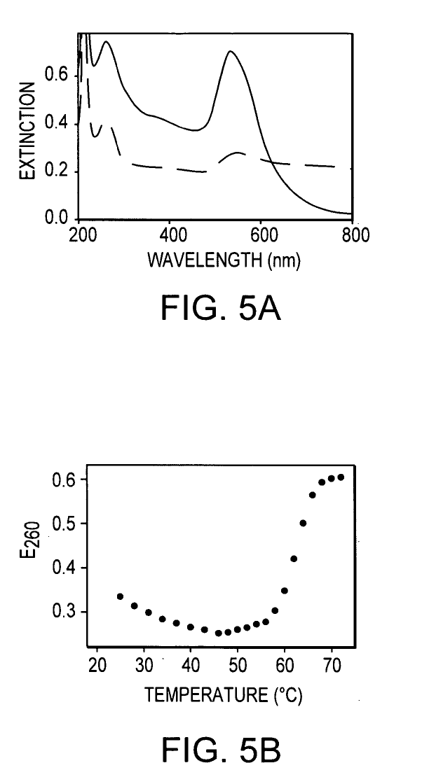

After allowing the ligation reaction to form substrate for approximately 30 minutes, the functionalized nanoparticles from Example 2 were added. The color change of the sample was monitored by UV-vis extinction spectroscopy. As shown by the solid line in FIG. 5A, the functionalized nanoparticles from Example 2 have an extinction peak at around 532 nm. In the presence of the substrate, the nanoparticles were assembled to form aggregates. Thus, upon aggregation, the extinction peak at around 532 nm decreased significantly while the extinction peak at around 700 nm increased, establishing the red-to-blue color transition of aggregate formation. Therefore, the extinction ratio at 532 and 700 nm was used to quantify the color of the system, with a higher ratio associated with the red color of separated particles, and a lower ratio associated with the blue color of aggregated particles.

### Example 7

### Confirmation that Aggregate Formation is Responsive to Substrate Formation

To confirm that aggregation and the associated red to blue color change was due to the ligated substrate, the melting properties of the aggregates were investigated. It is known that the extinction at 260 nm will increase significantly upon melting for DNA-assembled nanoparticles. See Elghanian, et al. Science (Washington, D.C.), 277, 1078-80 (1997) and jin, et al. J. Am. Chem. Soc. 125, 1643-54 (2003). FIG. 5B relates the extinction coefficient at 260 nm for the aggregates as temperature is increased. With the initial temperature increase, the extinction at 260 nm decreased, likely due to the formation of larger aggregates from smaller aggregates. For a more detailed description of this phenomenon, see Storhoff, et al., J. Am. Chem. Soc., 122, 4640-50 (2000). At higher temperature, the aggregates started to melt with a sharp increase of the extinction at 260 nm, consistent with the melting properties of nanoparticle aggregates assembled by ligated substrates.

### Example 8

### Confirming the Sensitivity of the Sensor System

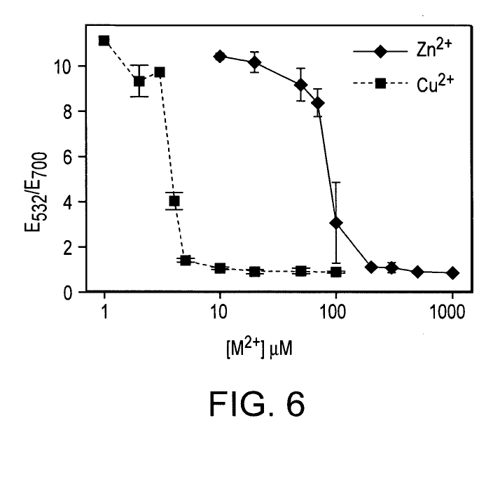

FIG. 6 depicts the change in extinction ratios for various concentrations of Cu(II) and Zn(II). It is clear from the graph that the sensor detects and provides a meaningful color change for Cu(II) at solution concentrations of from 3 to 10 μM and for Zn(II) at solution concentrations of from 20 to 200 μM. The extinction ratio decreased with the increase of metal concentrations for both metal ions. The middle point for Cu(II) was about 4 μM while the middle point for Zn(II) was about 80 μM. Thus, the ability of the sensor system to provide accurate quantitative information was established, with an approximately 20× heightened sensitivity to Cu(II) over Zn(II). These color changes may be visually observed by spotting onto an alumina TLC plate, for example.

### Example 9

### Confirming the Selectivity of the Sensor System

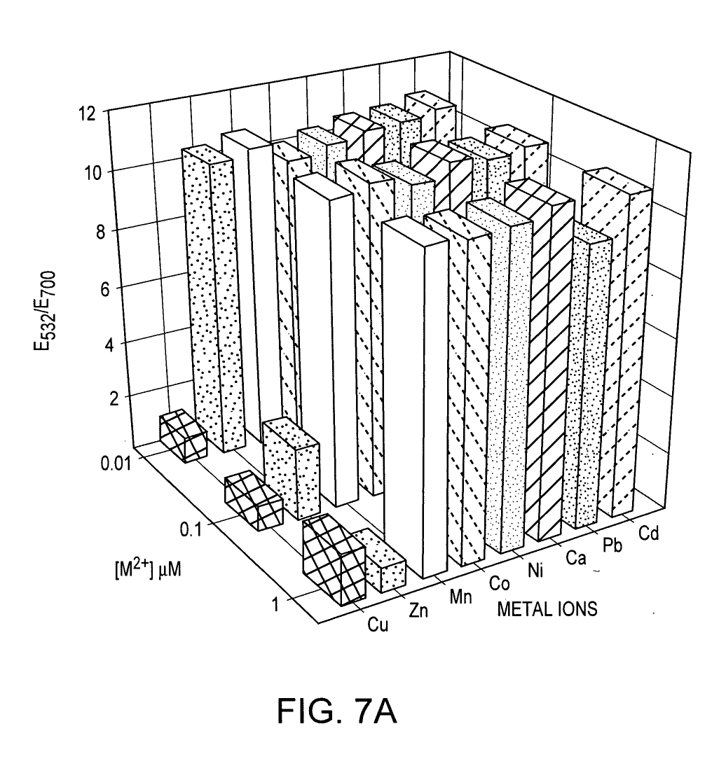

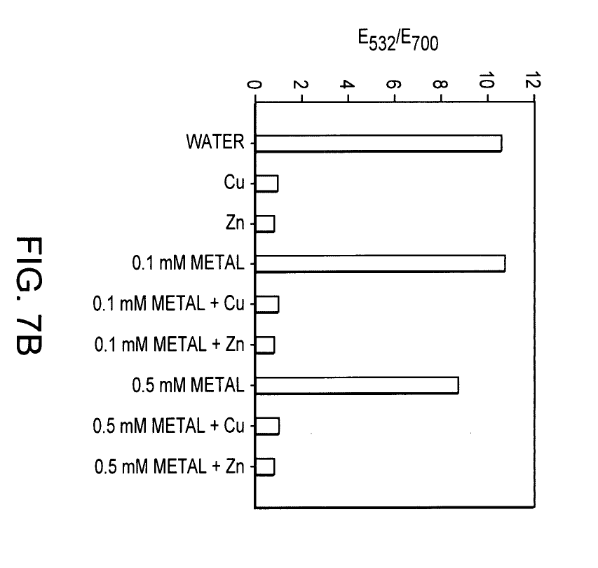

FIG. 7A depicts the extinction ratios for Cu(II), Zn(II), Mn(II), Co(II), Ni(II), Ca(II), Pb(II), and Cd(II) at the 0.01, 0.1, and 1 mM solution concentrations. The results establish that for the ions tested, significant changes in the extinction ratios were observed for Cu(II) and Zn(II), and for Zn(II) above the 0.01 mM concentration.

These results were further confirmed in FIG. 7B when a “metal soup” was supplied to the sensor that contained 0.1 mM or 0.5 mM of each of Mn(II), Co(II), Ni(II), Ca(II), Pb(II), and Cd(II). No color change was observed with the 0.1 mM metal soup and little was observed for the 0.5 mM soup. However, when 10 μM Cu(II) or 1 mM Zn(II) was added to the sensor containing the 0.1 mM or 0.5 mM metal soup, a color change was observed. The decrease in the extinction ratio with the 0.5 mM metal soup is believed attributable to the irreversible aggregation of gold nanoparticles induced by the high concentration of heavy metal ions; an effect not related to ligation.

### Example 10

### Confirming the Performance of the DNA aptazyme Sensor

The DNA aptazyme 225 as depicted in FIG. 2B was obtained from Integrated DNA Technologies. The following procedure was followed to test the ligation activity of this adenosine-dependent aptazyme. In a microcentrifuge tube, 39 μL of a 1.55 μM solution of the imidazole-activated substrate fragment depicted as 233 (FIG. 2B), 3.4 μL of a 17.7 μM solution of a 3′-fluorescein-labeled version of the substrate fragment depicted as 235 (FIG. 1B), 0.6 μL of a 100 μM solution of the DNA aptazyme 225 (FIG. 2B), 6 μL of 3 M NaCl, 3.6 μL of 500 mM HEPES buffer (pH 7.0), and 3.45 μL of deionized water were mixed. The total solution volume was 54 μL.

The 54 μL of solution was divided into two tubes with 27 μL in each tube. For the control tube, 3 μL of water was added, while for the other tube, 3 μL of 50 mM adenosine was added. Therefore, the final reagent concentration was 300 mM NaCl, 30 mM HEPES (pH 7.0), and 1 μM for the three DNA strands, and 5 mM adenosine for the non-control tube. After allowing the two tubes to sit at room temperature for 10 minutes, 1 μL of 30 mM ZnCl₂ was added to initiate the ligation reaction.

At designated time points, 5 μL aliquots were taken from each tube and transferred to other tubes containing EDTA to stop the reactions. When all time points were taken, the samples were loaded onto a 20% denaturing polyacrylamide gel, which separated the ligated substrates from the un-ligated substrate fragments. The gel was analyzed by exciting the fluorescein tag at 473 nm. Band intensity was quantified with Image Gauge software (Fuji).

As any person of ordinary skill in the art will recognize from the provided description, figures, and examples, that modifications and changes can be made to the preferred embodiments of the invention without departing from the scope of the invention defined by the following claims and their equivalents.

1. A sensor system for detecting an analyte, comprising:
a ligase;
a plurality of substrate fragments comprising first polynucleotides, at least two of the substrate fragments at least partially complementary to the ligase; and
first particles comprising second polynucleotides, the second polynucleotides coupled to the first particles, where the first polynucleotides are at least partially complementary to the second polynucleotides.

2. The sensor of claim 1, where at least two of the substrate fragments undergo ligation in the presence of the analyte.

3. The sensor of claim 1, further comprising second particles comprising third polynucleotides, the third polynucleotides coupled to the second particles at the 5′-terminus, where
the plurality of substrate fragments further comprise fourth polynucleotides having a different base sequence than the first polynucleotides, and
the second polynucleotides are coupled to the first particles at the 3′-terminus and the fourth polynucleotides are at least partially complementary to the third polynucleotides.

4. The sensor of claim 1, where the ligase is selected from the group consisting of nucleic acid enzymes, protein enzymes, small molecule mimics of ligase enzymes, and combinations thereof.

5. The sensor of claim 1, where the ligase comprises an aptamer, the aptamer comprising a fifth polynucleotide.

6. The sensor of claim 1, where the first particles comprise an inorganic material.

7. The sensor of claim 1, where the first particles comprise a material selected from the group consisting of metals, semiconductors, magnetizable materials, and combinations thereof.

8. The sensor of claim 3, where the first particles and the second particles comprise gold.

9. The sensor of claim 1, where the first particles have an average diameter from 5 nm to 100 nm.

10. The sensor of claim 1, where the first particles have an average diameter from 25 nm to 75 nm.

11. The sensor of claim 5, where the analyte binds with the aptamer to activate or deactivate the ligase in the presence of a co-factor.

12. The sensor of claim 1, where the analyte is selected from the group consisting of Ag(I), Pb(II), Hg(II), As(III), Fe(III), Zn(II), Cd(II), Cu(II), Sr(II), Ba(II), Ni(II), Co(II), As(V), U(VI), and Cr(VI).

13. The sensor of claim 1, where the analyte comprises a metal ion having a ⁺2 formal oxidation state.

14. The sensor of claim 1, where the analyte is selected from the group consisting of Cu(II), Zn(II), and combinations thereof.

15. The sensor of claim 11, where the analyte is selected from the group consisting of organic dyes, biotin, theophylline, adenine, dopamine, amino acids, nucleosides/nucleotides, RNA, biological co-factors, amino-glycosides, oligosaccharides, polysaccharides, peptides, enzymes, growth factors, transcription factors, antibodies, gene regulatory factors, cell adhesion molecules, cells, viral components, bacterial components, NH₄⁺, spermine, spermidine, adenosine, HIV, HIV proteins, HIV-derived molecules, anthrax, anthrax-derived molecules, small pox, small pox-derived molecules, nitrogen fertilizers, pesticides, dioxins, phenols, 2,4-dichlorophenoxyacetic acid, nerve gases, TNT, DNT, glucose, insulin, hCG-hormone, drugs, antibiotics, controlled substances, and cocaine.

16. The sensor of claim 11, where the analyte is selected from the group consisting of K(I), Zn(II), Ni(II), organic dyes, biotin, theophylline, adenine, dopamine, amino acids, nucleosides/nucleotides, RNA, biological co-factors, amino-glycosides, oligosaccharides, polysaccharides, peptides, enzymes, growth factors, transcription factors, antibodies, gene regulatory factors, cell adhesion molecules, cells, viral components, bacterial components, and cocaine.

17. The sensor of claim 1, where the ligase comprises a nucleic acid enzyme comprising a polynucleotide having a sequence selected from the group consisting of SEQ ID NO: 1, SEQ ID NO: 2, and conservatively modified variants thereof.

18. The sensor of claim 17, where the first polynucleotides comprise a plurality of polynucleotide fragments, each having a sequence selected from the group consisting of SEQ ID NO: 3, SEQ ID NO: 4, conservatively modified variants thereof, and combinations thereof.

19-20. (canceled)

21. A method of detecting an analyte, comprising:
combining a sample, a plurality of substrate fragments comprising first polynucleotides, and first particles comprising second polynucleotides, the second polynucleotides coupled to the first particles; and
detecting a color change responsive to the analyte, where
the first polynucleotides are at least partially complementary to the second polynucleotides and
at least two of the substrate fragments undergo ligation in the presence of the analyte.

22-39. (canceled)

40. A kit for detecting an analyte, comprising:
a system for forming aggregates, comprising:
a plurality of substrate fragments comprising first polynucleotides, where at least two of the plurality of substrate fragments undergo ligation in the presence of the analyte; and
first particles comprising second polynucleotides, the second polynucleotides coupled to the first particles, where
the first polynucleotides are at least partially complementary to the second polynucleotides.
at least one first container containing the system.

41-52. (canceled)
